(19) United States
(12) Patent Application Publication
(10) Pub. No.: US 2025/0308634 A1
(43) Pub. Date: Oct. 2, 2025

(54) OPTIMIZING PRIMER DESIGN IN RESTRICTION AND LIGATION-INDEPENDENT POLYMERASE CHAIN REACTION (PCR) CLONING

(71) Applicant: Virginia Commonwealth University, Richmond, VA (US)

(72) Inventors: Pradip Kumar Singh, Richmond, VA (US); Michael S. Donnenberg, Baltimore, MD (US)

(21) Appl. No.: 19/090,818

(22) Filed: Mar. 26, 2025

Related U.S. Application Data
(60) Provisional application No. 63/569,930, filed on Mar. 26, 2024.

(51) Int. Cl. G16B 25/20; G16B 30/00

(52) U.S. Cl. CPC G16B 25/20; G16B 30/00

(57) ABSTRACT

Disclosed herein are various embodiments for optimizing primer design in restriction and ligation-independent polymerase chain reaction cloning. First, a selection of a class is received from a client application. Then, a sequence input is received from the client application. Next, the sequence input is processed. Subsequently, a plurality of constraints are determined based at least in part on the processed sequence input. Lastly, a primer based at least in part on the plurality of constraints is designed.

### CROSS-REFERENCE TO RELATED APPLICATIONS

This application claims priority to and the benefit of U.S. Provisional Application No. 63/569,930, filed Mar. 26, 2024, which is hereby incorporated herein by reference in its entirety.

### SEQUENCE LISTING

This application contains a sequence listing filed in ST.26 format entitled “322203-1140 Sequences” created on Jun. 4, 2025 and having 41,763 bytes. The content of the sequence listing is incorporated herein in its entirety.

### BACKGROUND

Molecular cloning, a fundamental pillar of diverse biological research, often involves laborious multi-step procedures demanding significant time and resources. In a standard molecular cloning process, deoxyribonucleic acid (DNA) from an organism of interest is obtained and cleaved at specific site through the use of various enzymes. Once fragmented, the DNA is recombined with a cloning vector to produce recombinant DNA. The recombinant DNA can be introduced into a host organism to be reproduced or cloned.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIGS. 1A and 1B are example schematics for class 1 primer designing according to various embodiments of the present disclosure. FIG. 1A shows a vector and FIG. 1B shows an insert region primer design scheme. Shown in FIG. 1A are selected bases for the forward and reverse primers with the arrows indicating an insertion point or region. Note: in FIG. 1B, box f1c and r1c are the exact reverse complement of box f1 and r1 respectively, from FIG. 1A. The size of all other boxes gets adjusted according to Tm values. FIGS. 1C and 1D show class 2 primer designing schemes according to various embodiments of the present disclosure. FIG. 1C demonstrates that, for deletion primer design, sequences from region box fa & fb should be complementary to box rb and ra respectively and total length of each complementary boxes should be ˜8 bp. FIG. 1D shows an example of deletion and insertion primer design. In some embodiments, the sequence from box i-fa and i-ra can have a 16 base pair complementary sequence. The arrows mark the region of primers sequence and the “#” symbol indicates where FastCloneAssist tool adjusts respective sequence length to adjust Tm values.

FIG. 2 is a drawing of a network environment according to various embodiments of the present disclosure.

FIG. 3 is a flowchart illustrating one example of functionality implemented as portions of an application executed in a computing environment in the network environment of FIG. 2 according to various embodiments of the present disclosure.

FIG. 4 is a diagram showing the DNA and protein sequence of BfpE (SEQ ID NO: 29 (DNA) and SEQ ID NO: 39 (AA)), the linker L0 and His tag at N-terminus and C-terminus.

FIG. 5 is a diagram showing the DNA and protein sequence of interest of BfpD (SEQ ID NO: 30 (DNA) and SEQ ID NO: 40 (AA)), BfpU (SEQ ID NO: 31 (DNA) and SEQ ID NO: 41 (AA)) and BfpB (SEQ ID NO: 32 (DNA) and SEQ ID NO: 42 (AA)) and the target deletion is selected by arrows. Cloning sites of pET15TEV (SEQ ID NO: 33) and BfpD (N-Term) (SEQ ID NO: 34) are shown as well.

### DETAILED DESCRIPTION

Disclosed are various approaches for optimizing primer design in restriction and ligation-independent polymerase chain reaction (PCR) cloning. FastCloning is a widely-used cloning technique for plasmid reconstruction, allowing for a greatly simplified method for inserting any DNA fragment into a plasmid vector or into a gene. This paradigm shift in PCR cloning, has streamlined the process by eliminating laborious, multi-step traditional methods. FastCloning utilizes overlapping PCR primers and DpnI digestion for seamless integration of insert DNA into any desired vector position, regardless of restriction sites. This versatility makes FastCloning ideal for constructing fusion proteins, chimeric cDNAs, and manipulating genes with unparalleled ease.

However, efficient primer design can be a significant hurdle, particularly for newcomers, as errors can lead to failed cloning attempts. To address this bottleneck, this disclosure presents FastCloneAssist, a user-friendly Python™ program that can automate FastCloning primer design with minimal user input. Users simply provide a vector and insert sequences, along with the desired melting temperature (Tm), and FastCloneAssist can calculate the optimal primer parameters for efficient amplification and seamless integration using established bioinformatics libraries. This tool can simplify and accelerate FastCloning, making this a powerful technique accessible to researchers of all levels and expediting scientific discovery.

Molecular cloning can often involve laborious multi-step procedures demanding significant time and resources. Fortunately, FastCloning emerged as a transformative PCR-based approach, eliminating the need for restriction enzymes and ligation while fostering rapid fragment integration. This method relies on specifically designed primers for amplifying both vector and insert DNA, followed by DpnI treatment to selectively digest parental templates, and facilitate in vivo ligation (mechanism remains under investigation).

While FastCloning offers undeniable advantages, meticulous primer design remains crucial for successful amplification and seamless integration of the target fragment. Traditional strategies can often involve laborious manual calculations and sequence adjustments to attain optimal primer Tm, length, and overlap sequences, with a single base error potentially jeopardizing the entire cloning project. Moreover, existing online tools often lack customization options and can require coding expertise, limiting their accessibility to novice users.

To address these challenges, this disclosure introduces FastCloneAssist, a user-friendly Python™ script designed to streamline FastCloning primer design. Requiring minimal user input (vector and insert sequences), FastCloneAssist can automate the selection of optimal primer lengths, melting temperatures, and overlap sequences, ensuring efficient amplification and in vivo ligation.

Among other features and benefits of the present disclosure, FastCloneAssist can provide automated optimization for optimizing primer lengths, melting temperatures, and overlap sequences based at least in part on user-provided sequences. This can eliminate tedious manual calculations. Additionally, FastCloneAssist offers both default and user-defined Tm options, allowing for flexibility in primer design. Another benefit of the system is that it can require no coding knowledge, making it accessible to users of all skill levels. The platform independence of this invention also means that it can run easily on local computers in the Python™ environment and in the cloud. By streamlining primer design and overcoming technical barriers, FastCloneAssist can empower researchers to leverage the full potential of FastCloning, potentially accelerating diverse molecular biology applications.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principles disclosed by the following illustrative examples.

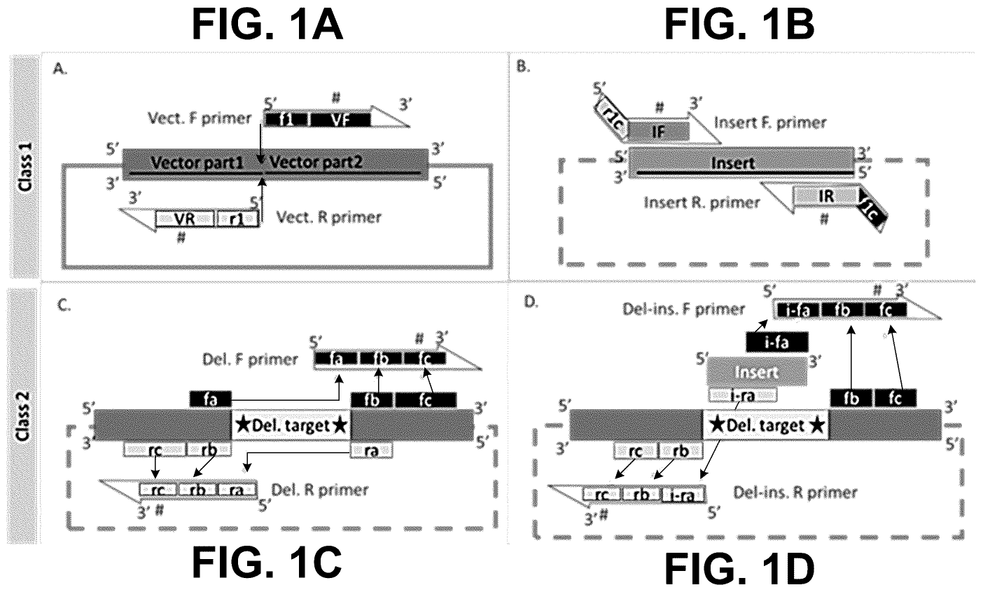

The scheme of primer designing is shown in FIGS. 1A-1D. The FastCloneAssist basic method can be divided into two classes. The first method is Class 1, which generates primer pairs for a chimera creation where an insert DNA (>100 bp) will be PCR amplified and inserted into a PCR linearized vector DNA. Class 1 method generates a total of two pairs of primers, one pair for vector and one for insert DNA. The second method is Class 2, which generates a primer pair which can be used to delete a region or insert or delete and insert. According to some examples, the insert should be less than 120 bp. Both Class 1 and Class 2 will be described in further detail below.

In some examples, the tool described herein uses the following libraries for the listed functions:
Biopython: can provide tools for sequence manipulation and translation.
   Biopython>SeqUtils.MeltingTemp: has specific capabilities for Tm calculations.
primer3: can be used for primer design and Tm calculations.
Bio.SeqUtils.Melting Temp: has specific capabilities for Tm calculations.
Tkinter: can be used to create the optional GUI interface.

### Inputs and Sequence Processing

To begin, a user can provide one or more inputs to the system. A first input can be a sequence. In some embodiments, for class 1, a user can provide a sequence of their desired chimera arrangement. For example, the user can provide the sequence in the format: vector_part-1 (5′ side to the insert's vector sequence [>40 bp])+insert sequence+vector_part-2 (3′ side to the insert's vector sequence [>40 bp])). According to various examples, these three pieces of the sequence must be separated or marked by “+” symbol (e.g., vector part 1+insert+vector part 2). According to various examples, the “vector part 1” and “vector part 2” should not be less that 40 bp. In some embodiments, the “vector part 1” and “vector part 2” should not be less than 50 bp, 70 bp, 100 bp, or 150 bp. Below is an example input sequence format for Class 1 (SEQ ID NO: 35):

TTGGCTGAAGCGATGAGGGGGTGGGTACCTTTTGACGAACTGTCAATAAT
TTCCGCCGGGGAAATCTCAGGTAATGTTCACCAGGCGTTGGATGATATCA
TTTATATGAATGATACAAAAAAGAAAGTAAAA
+
gcgcactggcagggattatttatcctgtagtcctgcttctgacgacatgt
ctgtatttgcatatatttggaactcaggttgttccggcattttcaggcat
cctgcctgtagagaaatggcagggcgcaggcaggactatgtattatcttg
ctgtattcgttcaggattatcttgtcattacactgctgtcttttatgatg
gtgatattattaatactggca
+
ACTCTGTCAAGATGGACCGGAAGGTTGCGTCTCTTCTTTGACAGATTTAT
TCCCTGGTCGATATACAAAACCATTATAGGATGTGGTTTCTTGTTATCAC
TGGCATCGCTTATTAATGCAGGTATCCCTGTACCGG

The insert sequence is in lower case and joined by “+” symbols into the vector part 1 and vector part 2 at the 5′ end and 3′ end of the insert sequence respectively.

In some embodiments, the user may also be asked to input a choice to customize the desired Tm range for primer design. In some embodiments, the system has a default range of, for example, approximately 55-65° C.

After receiving user inputs, the system can begin sequence processing. In some embodiments, the script splits the input sequence into specific parts as marked by the symbols (e.g., “+”). The system can identify and remove any gaps or line breaks from the sequences. Then, in some embodiments, the system can generate protein translation into one or more frames (e.g., −3, −2, −1, +1, +2, +3, etc.) for the final construct sequence.

Next, the system conducts a primer design step. In this step, the system can calculate the initial Tm values for potential primer sequences based at least in part on their nucleotide content. Then, the system can adjust primer lengths iteratively to achieve Tm values within the desired range (e.g., either the default or user-specified range). The system can determine the appropriate locations for primers based at least in part on the boundaries of the vector parts and insert.

In some embodiments, the system can design primers in Class 1 or in Class 2. To design primers for the vector amplification in Class 1, “vector_part1” and “vector_part2” sequences can be used. The “vector_part1” region specific primer can be designed by selecting nucleotides from 3′ ends toward 5′ of the sequence and the primer length adjusted to meet the desired Tm using SeqUtils modules from Biopython. The reverse complement of this sequence can be used as the reverse primer for vector. Forward primer for the vector can come from the “vector_part2” nucleotides which get selected from 5′ region of the sequence to meet the Tm and length of primer. Primers for the insert sequence can come from the 5′ and 3′ terminals of the insert sequence and get a 16 bp complementary overhang from respective vector regions. The forward primer of the insert (FPI) get overhang from vector_part1 whereas reverse primer of the insert (RPI) get overhang from the vector_part2, see the FIG. 1. The length of FPI and RPI (without their overhangs) also get adjusted to meet the desired Tm values.

For Class 2 primer design, the system can design primers for deletion, insertion and/or deletion & insertion. In some embodiments, a user is asked to provide DNA sequences in a format where the deletion region (start & end) will be marked by “*” symbols and the insert sequence will be provided at the end of sequence marked by another “*” symbol. One example, demonstrating the format of the input, is provided below (SEQ ID NO: 36):

ACTCTGTCAAGATGGACCGGAAGGTTGCGTCTCTTCTTTGACAGATTTAT
TCCCTGGTCGATATACAAAACCATTATAGGATGTGGTTTCTTGTTATCA*
CTGGCATCGCTTATTAATGCAGGTATCCC*TGTACCGGAAGCCTTACGAA
TAATAATGAAAACGGCAAGT*ccgtggtataaggaaagattagttgcta

The deletion region is shown underlined and marked by “*” at the start and finish. Likewise, the insert sequence, in small case, is demarked by another “*” symbol.

In some embodiments, a user may wish to only make an insertion, without a deletion. In such examples, the user is asked to provide an input sequence by marking the insertion point by two stars “**” or another symbol, and to provide the insert sequence as above. One example, demonstrating the format of the input sequence, is shown below (SEQ ID NO: 37):

ACTCTGTCAAGATGGACCGGAAGGTTGCGTCTCTTCTTTGACAGATTTAT
TCCCTGGTCGATATACAAAACCATTATAGGATGTGGTTTCTTGTTATCA*
*CTGGCATCGCTTATTAATGCAGGTATCCCTGTACCGGAAGCCTTACGAA
TAATAATGAAAACGGCAAGT*ccgtggtataaggaaagattagttgcta

For deletion-only primer designing, the input sequence format can be as follows: the deletion can be marked using a star (*) or other symbol, as described in the deletion insertion example. Further, the format can include placement of a third star at the end of sequence. One example, demonstrating the format of the deletion-only input sequence, is shown below (SEQ ID NO: 38):

ACTCTGTCAAGATGGACCGGAAGGTTGCGTCTCTTCTTTGACAGATTTAT
TCCCTGGTCGATATACAAAACCATTATAGGATGTGGTTTCTTGTTATCA*
CTGGCATCGCTTATTAATGCAGGTATCCC*TGTACCGGAAGCCTTACGAA
TAATAATGAAAACGGCAAGT*

In some embodiments, the system can produce one or more outputs. The system can generate and display a FASTA format of the final construct sequence. In some embodiments, the system generates and displays translations in all three frames (for example, when the optional translation feature is used). In addition, according to various examples, the system can output designed primer sequences, along with their Tm values and lengths. In some examples, the system can output a report saved as .txt file or other file format.

The FastCloneAssist tool can be run in a python environment installed locally. In some embodiments, a user can utilize the FastCloneAssist script by first downloading it from a GitHub repository and executing it using Python. According to various examples, the code is structured into three parts. A user can begin by running the first part of the code to install the required libraries (e.g., Biopython & primer3). In some embodiments, it is important to note that the first part only needs to be executed once for a system, as it installs the necessary libraries. Once this step is completed, the user can proceed to run the second part. The second part of the code can import all required modules from the installed libraries. Note the tkinter, sys, and os modules can be imported from the default Python installation.

Running the third part of the code (the primer design part), a user can be prompted to choose either Class 1 or Class 2 style of primer designing (see above). Following a selection, the next required input will be the sequence. Ensure the sequence is pre-prepared with symbols as described above for the specific class and need. The next prompts can ask the user if the user wants to choose any specific Tm or use the default range (55-65° C.)? The user can enter “no”, but if the user input is “yes,” the code will prompt the user to enter the Tm range, such as “60-64”. After this input, the script can generate a report as a text file. In some embodiments, the user will receive a popup asking the user to save the file.

In some embodiments, the FastCloneAssist tool can be run using google Colab. In such embodiments, no Python installation is required on the computing device. To use the tool in Google Colab, a user can first create a Colab account using a Google account. The user can download the Colab version of the script from a GitHub link and save it in your Google Drive. In Colab, the user can import the script from their Google Drive and run it. The user can right click on file “FastCloneAssist_Colab” file in their google drive and choose “run with Google Colaboratory.” Note, in Colab, a user may need to run the first part of the code each time they start a new session, as Google Cloud may not store the installed library once the session ends. The primer can be saved as a text file in the download folder.

While the above describes two nonlimiting examples of a user journey in using the FastCloneAssist tool, there are many other user experiences that could occur for the user to use FastCloneAssist. The tool FastCloneAssist streamlines and expedites Fast Cloning, enhancing accessibility for researchers of all levels. This advancement holds the promise of further democratizing molecular biology and accelerating the pace of scientific discovery.

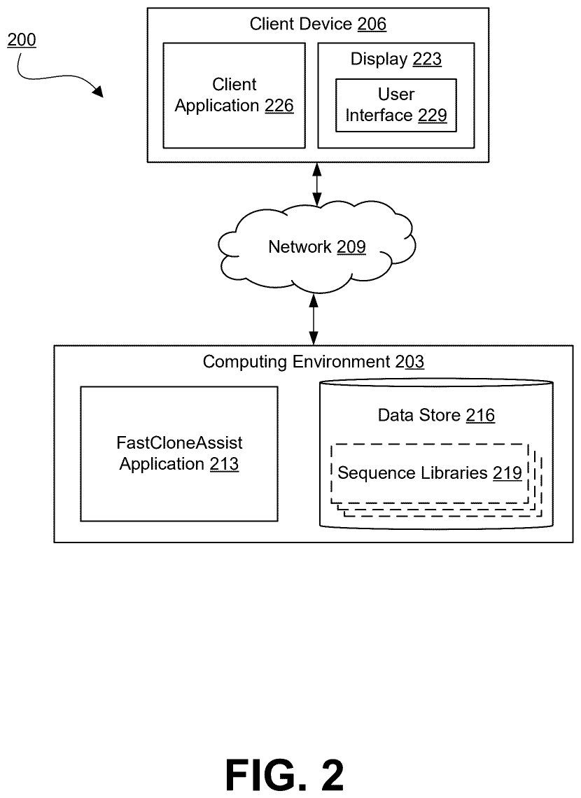

With reference to FIG. 2, shown is a network environment 200 according to various embodiments. The network environment 200 can include a computing environment 203 and a client device 206, which can be in data communication with each other via a network 209

The network 209 can include wide area networks (WANs), local area networks (LANs), personal area networks (PANs), or a combination thereof. These networks can include wired or wireless components or a combination thereof. Wired networks can include Ethernet networks, cable networks, fiber optic networks, and telephone networks such as dial-up, digital subscriber line (DSL), and integrated services digital network (ISDN) networks. Wireless networks can include cellular networks, satellite networks, Institute of Electrical and Electronic Engineers (IEEE) 802.11 wireless networks (i.e., WI-FI®), BLUETOOTH® networks, microwave transmission networks, as well as other networks relying on radio broadcasts. The network 209 can also include a combination of two or more networks 209 Examples of networks 209 can include the Internet, intranets, extranets, virtual private networks (VPNs), and similar networks.

The computing environment 203 can include one or more computing devices that include a processor, a memory, and/or a network interface. For example, the computing devices can be configured to perform computations on behalf of other computing devices or applications. As another example, such computing devices can host and/or provide content to other computing devices in response to requests for content.

Moreover, the computing environment 203 can employ a plurality of computing devices that can be arranged in one or more server banks or computer banks or other arrangements. Such computing devices can be located in a single installation or can be distributed among many different geographical locations. For example, the computing environment 203 can include a plurality of computing devices that together can include a hosted computing resource, a grid computing resource or any other distributed computing arrangement. In some cases, the computing environment 203 can correspond to an elastic computing resource where the allotted capacity of processing, network, storage, or other computing-related resources can vary over time.

Various applications or other functionality can be executed in the computing environment 203. The components executed on the computing environment 203 include the FastCloneAssist application 213, and other applications, services, processes, systems, engines, or functionality not discussed in detail herein.

The FastCloneAssist application 213 can be executed to receive one or more inputs from a client device 206. The FastCloneAssist application 213 can be executed to receive a selection of a class, designate a format for a sequence input, receive a sequence input and process the sequence input. The FastCloneAssist application 213 can determine a plurality of constraints, such as a melting temperature, a primer length, and/or an overlap sequence. Next, the FastCloneAssist application 213 can design a primer based at least in part on the plurality of constraints.

Also, various data is stored in a data store 216 that is accessible to the computing environment 203. The data store 216 can be representative of a plurality of data stores 216 which can include relational databases or non-relational databases such as object-oriented databases, hierarchical databases, hash tables or similar key-value data stores, as well as other data storage applications or data structures. Moreover, combinations of these databases, data storage applications, and/or data structures may be used together to provide a single, logical, data store. The data stored in the data store 216 is associated with the operation of the various applications or functional entities described below. This data can include sequence libraries 219, and potentially other data.

The sequence libraries 219 can represent any grouping or catalog of DNA fragments which have adapters attached. Examples of sequence libraries 219 include Biopython, which can provide tools for sequence manipulation and translation. In addition, sequence libraries may include the Biopython SeqUtils.MeltingTemp tool, which has specific capabilities for Tm calculations. Another example is Primer3, which can be used for primer design and Tm calculations; Bio.SeqUtils.MeltingTemp, which has specific capabilities for Tm calculations; Tkinter, which can be used to create the optional GUI interface; and many other sequence libraries, databases, or primer design tools.

The client device 206 is representative of a plurality of client devices that can be coupled to the network 209 The client device 206 can include a processor-based system such as a computer system. Such a computer system can be embodied in the form of a personal computer (e.g., a desktop computer, a laptop computer, or similar device), a mobile computing device (e.g., personal digital assistants, cellular telephones, smartphones, web pads, tablet computer systems, music players, portable game consoles, electronic book readers, and similar devices), media playback devices (e.g., media streaming devices, BluRay® players, digital video disc (DVD) players, set-top boxes, and similar devices), a videogame console, or other devices with like capability. The client device 206 can include one or more displays 223, such as liquid crystal displays (LCDs), gas plasma-based flat panel displays, organic light emitting diode (OLED) displays, electrophoretic ink (“E-ink”) displays, projectors, or other types of display devices. In some instances, the display 223 can be a component of the client device 206 or can be connected to the client device 206 through a wired or wireless connection.

The client device 206 can be configured to execute various applications such as a client application 226 or other applications. The client application 226 can be executed in a client device 206 to access network content served up by the computing environment 203 or other servers, thereby rendering a user interface 229 on the display 223. To this end, the client application 226 can include a browser, a dedicated application, or other executable, and the user interface 229 can include a network page, an application screen, or other user mechanism for obtaining user input. The client device 206 can be configured to execute applications beyond the client application 226 such as email applications, social networking applications, word processors, spreadsheets, or other applications.

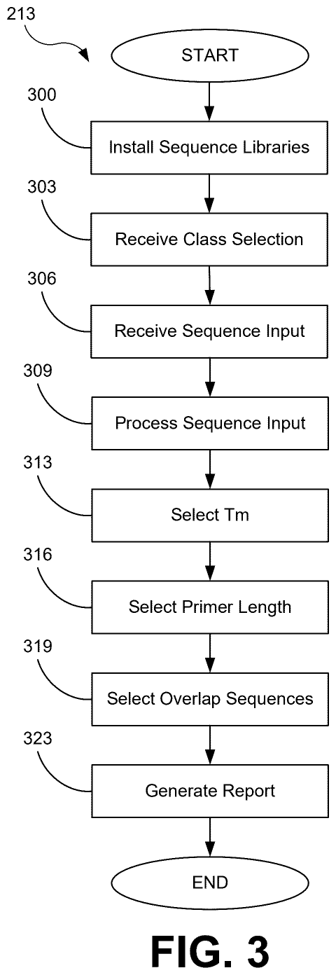

Moving to FIG. 3, shown is a flowchart that provides one example of the operation of a portion of the FastCloneAssist application 213. The flowchart of FIG. 3 provides merely an example of the many different types of functional arrangements that can be employed to implement the operation of the depicted portion of the FastCloneAssist application 213. As an alternative, the flowchart of FIG. 3 can be viewed as depicting an example of elements of a method implemented within the network environment 200.

Beginning with block 300, the FastCloneAssist application 213 can install one or more sequence libraries 219. In some embodiments, the FastCloneAssist application 213 can find and download the sequence libraries 219 from a computer, server, device, or other system connected over the network 209. In some embodiments, the FastCloneAssist application 213 can find and download the sequence libraries 219 from the internet. The FastCloneAssist application 213 can save the sequence libraries 219 in a data store 216 in the computing environment 203, or in another location.

At block 303, the FastCloneAssist application 213 can receive a class selection. The selection of a class can be a “1” input or a “2” input or another class selection input. In some embodiments, the FastCloneAssist application 213 receives a class selection from a client device 206 via the client application 226. The selection of a class can come from a user interaction with the user interface 229 of a client device 206 and be transmitted to the FastCloneAssist application 213 by the client application 226. In some embodiments, the FastCloneAssist application 213 receives a selection of a class from another source in the network environment 200.

Moving to block 306, the FastCloneAssist application 213 can receive a sequence input. The sequence input can be formatted according to the class selection received at block 303. In some embodiments, the FastCloneAssist application 213 can cause a prompt to appear on a user interface 229 of a client device 206 in response to receiving a class selection at block 303. The prompt can request a sequence input in a format corresponding to the class selection received at block 303. The user can provide a sequence input in the corresponding format, which can be transmitted from the client device 206 to the FastCloneAssist application 213 by the client application 226. In some embodiments, the FastCloneAssist application 213 receives the sequence input from another source in the network environment 200.

Next, at block 309, the FastCloneAssist application 213 can process the sequence input received at block 306. In some embodiments, the FastCloneAssist application 213 interprets the sequence input according to the format corresponding to the class selection received at block 303. The FastCloneAssist application 213 can split the sequence input into various different parts using the designated format. Additionally, in some embodiments, the FastCloneAssist application 213 can remove spaces, gaps, line breaks, or other miscellaneous formatting errors from the sequences. According to various examples, the FastCloneAssist application 213 can generate protein translation from the sequence input into one or more frames (e.g., +1, +2, +3, etc.) for the final construct sequence.

At block 313, the FastCloneAssist application 213 can begin designing a primer by selecting a melting temperature (Tm). In some embodiments, the FastCloneAssist application 213 can calculate an initial melting temperature for the potential primer sequences based at least in part on the nucleotide content of the input sequence received at block 306. In some embodiments, the FastCloneAssist application 213 selects the melting temperature based at least in part on the sequence input received at block 306 and based at least in part on the sequence libraries 219 installed at block 300. In some embodiments, the FastCloneAssist application 213 selects a melting temperature by generating a prompt to be presented to a user, the prompt requesting a melting temperature input. In some embodiments, the FastCloneAssist application 213 selects a melting temperature based at least in part on a response to such a prompt. For example, the FastCloneAssist application 213 can select a melting temperature based at least in part on a melting temperature input. In some embodiments, the FastCloneAssist application 213 can select a melting temperature from a default range (e.g., 55-65° C.).

Moving to block 316, the FastCloneAssist application 213 can select a primer length. In some embodiments, the FastCloneAssist application 213 can select a primer length iteratively with selecting a melting temperature at block 313. For example, the FastCloneAssist application 213 can adjust the selected primer length based at least in part on whether the melting temperature corresponding to the primer length is within the desired melting temperature range. In some embodiments, the FastCloneAssist application 213 can select a primer length based at least in part on the sequence libraries 219 installed at block 300, the class selection received at block 303, and/or the sequence input received at block 306.

Moving to block 319, the FastCloneAssist application 213 can select overlap sequences. The overlap sequences can be selected based at least in part on the primer sequences. As shown in FIGS. 1A & B for Class 1, the overlap sequences f1c and r1c are reverse complements of the primer sequences f1 and r1. In some embodiments, the optimized overlap sequence length is in the range of 15 to 17 base pairs (bp). In some embodiments, the optimized overlap sequence length is in the range of 14 to 18 bp. In some embodiments, the optimized overlap sequence length is 16 bp. Similarly, for Class 2, FIGS. 1C & D show that sequences fa and fb are complementary to sequences rb and ra respectively. In some embodiments, the optimized overlap sequence length for Class 2 is approximately 8 base pairs for each box (e.g., fa, fb, rb, ra, etc.).

Next, at block 323, the FastCloneAssist application 213 can generate a report. The report can comprise a notification, message, text file, image file, link, or other report which can be used to identify the primer designed by the FastCloneAssist application 213. According to various examples, the report can include a FASTA format of the final construct sequence, translations in all three frames, the designed primer sequences along with their respective melting temperatures and lengths, etc. In some embodiments, the FastCloneAssist application 213 can generate the report based at least in part on the selected melting temperature from block 313, the selected primer length from block 316, and/or the selected overlap sequences from block 319. In some embodiments, the FastCloneAssist application 213 can send the report to a client device 206 to be displayed on a display 223 of the client device 206. After block 323, the flowchart of FIG. 3 ends.

A number of software components previously discussed are stored in the memory of the respective computing devices and are executable by the processor of the respective computing devices. In this respect, the term “executable” means a program file that is in a form that can ultimately be run by the processor. Examples of executable programs can be a compiled program that can be translated into machine code in a format that can be loaded into a random access portion of the memory and run by the processor, source code that can be expressed in proper format such as object code that is capable of being loaded into a random access portion of the memory and executed by the processor, or source code that can be interpreted by another executable program to generate instructions in a random access portion of the memory to be executed by the processor. An executable program can be stored in any portion or component of the memory, including random access memory (RAM), read-only memory (ROM), hard drive, solid-state drive, Universal Serial Bus (USB) flash drive, memory card, optical disc such as compact disc (CD) or digital versatile disc (DVD), floppy disk, magnetic tape, or other memory components.

The memory includes both volatile and nonvolatile memory and data storage components. Volatile components are those that do not retain data values upon loss of power. Nonvolatile components are those that retain data upon a loss of power. Thus, the memory can include random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, or other memory components, or a combination of any two or more of these memory components. In addition, the RAM can include static random access memory (SRAM), dynamic random access memory (DRAM), or magnetic random access memory (MRAM) and other such devices. The ROM can include a programmable read-only memory (PROM), an erasable programmable read-only memory (EPROM), an electrically erasable programmable read-only memory (EEPROM), or other like memory device.

Although the applications and systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowchart shows the functionality and operation of an implementation of portions of the various embodiments of the present disclosure. If embodied in software, each block can represent a module, segment, or portion of code that includes program instructions to implement the specified logical function(s). The program instructions can be embodied in the form of source code that includes human-readable statements written in a programming language or machine code that includes numerical instructions recognizable by a suitable execution system such as a processor in a computer system. The machine code can be converted from the source code through various processes. For example, the machine code can be generated from the source code with a compiler prior to execution of the corresponding application. As another example, the machine code can be generated from the source code concurrently with execution with an interpreter. Other approaches can also be used. If embodied in hardware, each block can represent a circuit or a number of interconnected circuits to implement the specified logical function or functions.

Although the flowchart shows a specific order of execution, it is understood that the order of execution can differ from that which is depicted. For example, the order of execution of two or more blocks can be scrambled relative to the order shown. Also, two or more blocks shown in succession can be executed concurrently or with partial concurrence. Further, in some embodiments, one or more of the blocks shown in the flowchart can be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein that includes software or code can be embodied in any non-transitory computer-readable medium for use by or in connection with an instruction execution system such as a processor in a computer system or other system. In this sense, the logic can include statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. Moreover, a collection of distributed computer-readable media located across a plurality of computing devices (e.g., storage area networks or distributed or clustered filesystems or databases) may also be collectively considered as a single non-transitory computer-readable medium.

The computer-readable medium can include any one of many physical media such as magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium can be a random access memory (RAM) including static random access memory (SRAM) and dynamic random access memory (DRAM), or magnetic random access memory (MRAM). In addition, the computer-readable medium can be a read-only memory (ROM), a programmable read-only memory (PROM), an erasable programmable read-only memory (EPROM), an electrically erasable programmable read-only memory (EEPROM), or other type of memory device.

Further, any logic or application described herein can be implemented and structured in a variety of ways. For example, one or more applications described can be implemented as modules or components of a single application. Further, one or more applications described herein can be executed in shared or separate computing devices or a combination thereof. For example, a plurality of the applications described herein can execute in the same computing device, or in multiple computing devices in the same computing environment 203

### Example 1

The FastCloning method was useful in a mutagenesis project involving the creation of various chimera genes and mutants to explore the biophysical properties of a set of proteins, BfpE, BfpD, BfpC, BfpB and BfpU from the enteropathogenic E. coli type IV pilus, known as the Bundle forming pilus (BFP). The FastCloning method reduced the total time and cost of chimera/mutant creation. However, manual design and verification of FastCloning primers required considerable additional effort. The FastCloneAssist, as disclosed herein, significantly improved primer designing capabilities and primer quality.

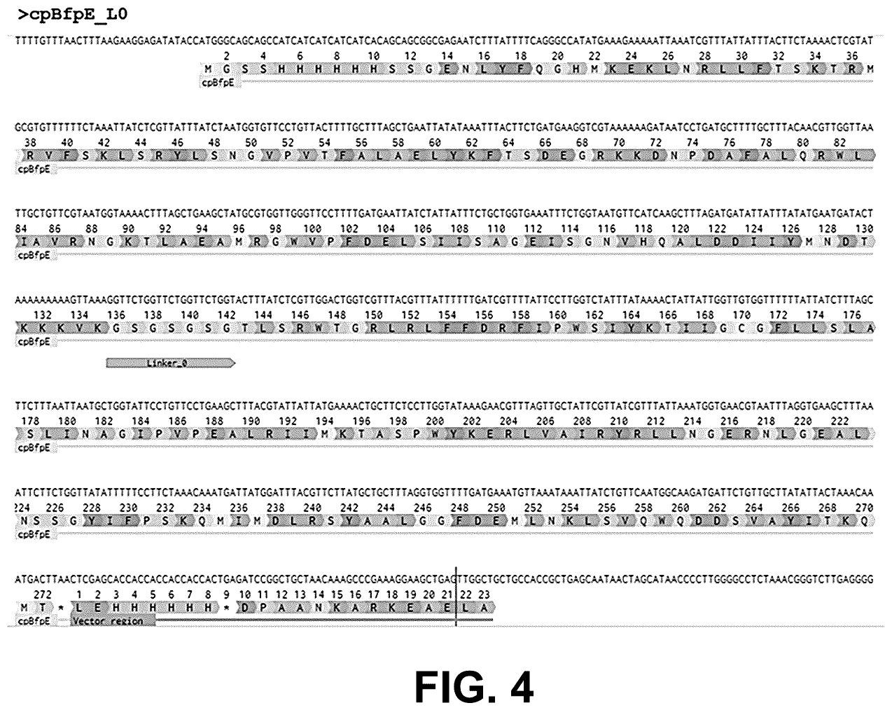

In the case of BfpE, a recombinant protein (cpBfpE) had been created by deleting transmembrane regions in an attempt to express a soluble protein. However, protein expression was insufficient for purposes of the project. To troubleshoot expression, various versions needed to be cloned, altering the linker (L) length and sequences, deletion of some more sequences (T143-K165) that could represent additional transmembrane sequences, and changing the His tag location from N-term to C-term. For all of these events, the FastCloneAssist tool was used to design the primers and perform the cloning effectively. FIG. 4 shows the cpBfpE sequence and translation. The linker region L0 has been changed with L1, L2, L3 and L5. The linker sequences are listed below, and primers (1-14) are shown in Table 1.

L1 =
SEQ ID NO: 1
5′ ggt tct cca ggt tct 3′; (GSPGS)
L2 =
SEQ ID NO: 2
5′ ggt tct cca ggt cca agc ggt 3′; (GSPGPGG)
L3 =
SEQ ID NO: 3
5′ ggt ggc ggt ggt tct 3′; (GGGGS)
L4 =
SEQ ID NO: 4
5′ ggt tct gct ggt tct gct gct ggt tct ggt gaa ttc
3′; (GSAGSAAGSGEF)

The parenthesis contains the corresponding protein sequence.

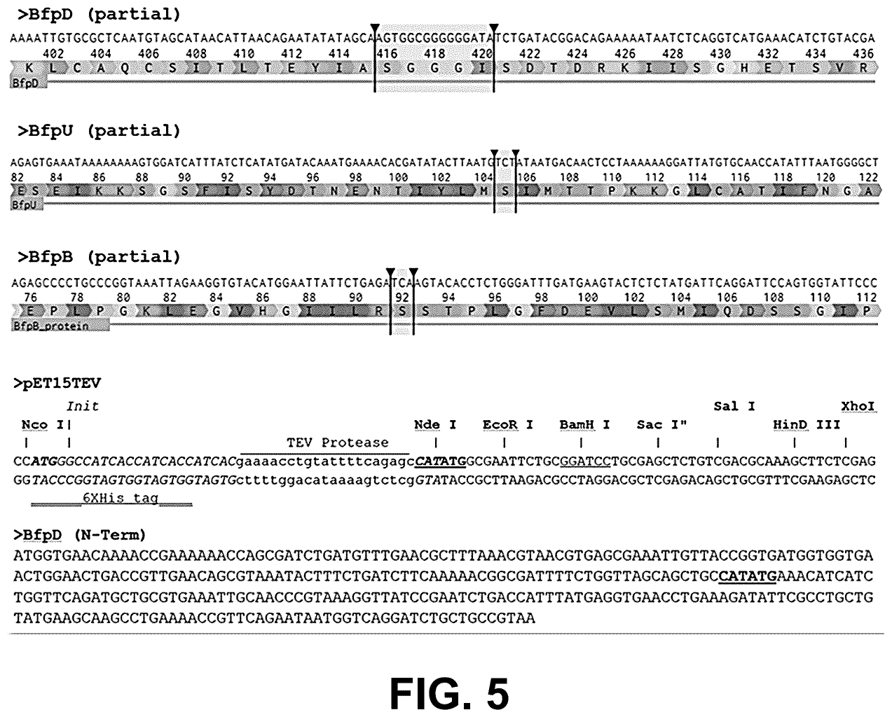

In a similar approach (deletion and insertion), FastCloneAssist was used to insert a FLASH tag (CCPGCC) in the BfpD protein at the specific location (416-420). See FIG. 5 and Table 1, primers 15-16.

FastCloning and FastCloneAssist were also used to create a point mutation in BfpU and BfpB at S105C and S92C respectively, see FIG. 5 and Table 1, primers 17-20.

To ligate N-term BfpD into pET15TEV vector and avoid an additional restriction (NdeI) site present in the middle of the gene (N-term BfpD), FastCloning was used and primers (Table 1, primers 21-24) were designed using FastCloneAssist (see FIG. 5).

FastCloning and FastCloneAssist revolutionized the approach to chimera and mutant creation, accelerating experimentation and enhancing precision. This example illustrates the efficacy of these tools in facilitating complex genetic manipulations, paving the way for advancements in protein engineering and biophysical studies.

TABLE 1
List of primers designed using FastCloneAssist tool.

| SEQ | Primer | | |
|---|---|---|---|
| ID No: | name | Sequence (5′ → 3′) | Purpose |
| 5 | cpBfpE_L1f | ggttctccaggttctACTTTATCTCGTTGGACTGGT | Del^{a}/ins^{b}; |
| | | CGTTTA | Primer |
| 6 | cpBfpE_L1r | TagaacctggagaaccTTTAACTTTTTTTTTAGTAT | pair to |
| | | CATTCATATAAATAATATCATCTAAAGC | delete L0 |
| | | | and add |
| | | | L1 |
| 7 | cpBfpE_L2f | tctccaggtccaagcggtACTTTATCTCGTTGGACT | Del/ins; |
| | | GGTCGTTTA | Primer |
| 8 | cpBfpE_L2r | cgcttggacctggagaaccTTTAACTTTTTTTTTAG | pair to |
| | | TATCATTCATATAAATAATATCATCTAAAGC | delete L0 |
| | | | and add |
| | | | L2 |
| 9 | cpBfpE_L3f | ggtggcggtggttctACTTTATCTCGTTGGACTGGT | Del/ins; |
| | | CGTTTA | Primer |
| 10 | cpBfpE_L3r | TagaaccaccgccaccTTTAACTTTTTTTTTAGTAT | pair to |
| | | CATTCATATAAATAATATCATCTAAAGC | delete L0 |
| | | | and add |
| | | | L3 |
| 11 | cpBfpE_L4f | gttctgctgctggttctggtgaattcACTTTATCTC | Del/ins; |
| | | GTTGGACTGGTCGTTTA | Primer |
| 12 | cpBfpE_L4r | gaaccagcagcagaaccagcagaaccTTTAACTTTT | pair to |
| | | TTTTTAGTATCATTCATATAAATAATATCATCTAAA | delete L0 |
| | | GC | and add |
| | | | L4 |
| 13 | cpE_L0_del | GTTCTGGTACTATTATTGGTTGTGGTTTTTTATTAT | Pair to |
| | 187-211 F | CTTTAGCT | Delete |
| 14 | cpE_L0_del | ATAATAGTACCAGAACCAGAACCAGAACCTTTAAC | T143 - |
| | 187-211_R | | K165) in |
| | | | cpBfpE |
| 15 | cpE_C- | AAATGACTCTCGAGCACCACCACCACCACCACTG | Del; |
| | His_F | | Primer |
| 16 | cpE_C- | TGCTCGAGAGTCATTTGTTTAGTAATATAAGCAACA | pair to |
| | His_R | GAATCA | delete the |
| | | | stop |
| | | | codon |
| | | | after |
| | | | Cterm |
| | | | T272. |
| 17 | cpE_N- | GATATACCATGAAAGAAAAATTAAATCGTTTATTAT | Del: |
| | His_F | TTACTTCTAAAACTC | Primer |
| 18 | cpE_N- | TCTTTCATGGTATATCTCCTTCTTAAAGTTAAACAA | pair to |
| | His_R | AATTATTTCTAGA | delete the |
| | | | His_tag |
| | | | from |
| | | | Nterminus |
| 19 | PKS61F | gttgcccgggctgctgtTCTGATACGGACAGAAAAA | Primer |
| | | TAATCTCAGG | pair to |
| 20 | PKS61R | cagcagcccgggcaacaACTTGCTATATATTCTGTT | insert |
| | | AATGTTATGCTACATTG | FLASH |
| | | | tag at |
| | | | BfpD |
| | | | 416-420. |
| 21 | FC_BfpB_S9 | CTGAGATgtAGTACACCTCTGGGATTTGATGAAGTA | Prime pair |
| | 2C_F | | to replace |
| 22 | FC_BfpB_S9 | GTGTACTacATCTCAGAATAATTCCATGTACACCTT | S92 with |
| | 2C_R | CTAAT | C from |
| | | | BfpB |
| | | | protein. |
| 23 | FC_BfpU_S | TTAATGTgTATAATGACAACTCCTAAAAAAGGATTA | Prime pair |
| | 105C_F | TGTGC | to replace |
| 24 | FC_BfpU_S | TCATTATACACATTAAGTATATCGTGTTTTCATTTG | S105 with |
| | 105C_R | TATCATATGA | C from |
| | | | BfpU |
| | | | protein. |

Vector and insert PCR and Chimera creation

| | | | |
|---|---|---|---|
| 25 | PMD22_05- | CATATGgctctgaaaatacaggttttcGTGATG | Primer |
| | vR | | pair to |
| 26 | PMD22_06- | TAAGGATCCTGCGAGCTCTGTCGA | amplify |
| | vF | | vector |
| | | | pET15TEV |
| | | | to clone |
| | | | N-term |
| | | | BfpD. |
| 27 | PMD22_07- | ctgtattttcagagcCATATGGTGAACAAAACCGAA | Primer |
| | iF | AAAACCAGCG | pair to |
| 28 | PMD22_08- | GAGCTCGCAGGATCCTTACGGCAGCAGATCCTGACC | amplify |
| | iR | ATTATT | and clone |
| | | | the N- |
| | | | term BfpD |
| | | | into |
| | | | pET15TEV |

^{a}Del = deletion,
^{b}ins = insertion

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. All such modifications and variations are intended to be included herein within the scope of this disclosure and protected by the following claims.

Therefore, the following is claimed:

1. A system, comprising:
a computing device comprising a processor and a memory; and
machine-readable instructions stored in the memory that, when executed by the processor, cause the computing device to at least:
receive a selection of a class, wherein the class is a chimera creation class or a modification class;
receive a sequence input;
process the sequence input;
determine a plurality of constraints based at least in part on the processed sequence input; and
design a primer based at least in part on the plurality of constraints.

2. The system of claim 1, wherein the plurality of constraints includes at least one of a melting temperature, a primer length, and an overlap sequence.

3. The system of claim 1, wherein the machine-readable instructions further cause the computing device to at least generate a report including information on the primer.

4. The system of claim 1, wherein the selection of the class comprises the selection of a chimera creation class, and the machine-readable instructions further cause the computing device to establish a chimera creation format corresponding to the sequence input.

5. The system of claim 4, wherein the chimera creation format comprises “Vector Part 1+Insert+Vector Part 2.”

6. The system of claim 1, wherein the selection of the class comprises the selection of a modification class, and the machine-readable instructions further cause the computing device to establish a modification format corresponding to the sequence input.

7. The system of claim 6, wherein the modification format comprises a deletion format, an insertion format, or a combination format.

8. The system of claim 1, wherein the machine-readable instructions which cause the computing device to determine the plurality of constraints based at least in part on the processed sequence input, further cause the computing device to at least:
install one or more sequence libraries; and
determine the plurality of constraints based at least in part on the one or more sequence libraries.

9. The system of claim 8, wherein the machine-readable instructions which cause the computing device to process the sequence input further cause the computing device to compare the sequence input to the one or more sequence libraries.

10. The system of claim 1, wherein the machine-readable instructions which cause the computing device to determine a plurality of constraints further cause the computing device to:
receive a desired range of melting temperatures; and
design the primer based at least in part on the plurality of constraints and the desired range of melting temperatures.

11. The system of claim 1, wherein the machine-readable instructions which cause the computing device to process the sequence input further cause the computing device to:
split the sequence input into two or more parts based at least in part on one or more symbols in the sequence input; and
generate a protein translation into one or more frames.

12. The system of claim 1, wherein the machine-readable instructions which cause the computing device to design the primer further cause the computing device to:
receive a desired range of melting temperatures;
calculate a melting temperature value for one or more primers based at least in part on a plurality of nucleotides within the one or more primers;
adjust a length of one or more primers based at least in part on the calculated melting temperature value;
determine the calculated melting temperature value is within the desired range; and
determine one or more appropriate locations for the one or more primers based at least in part on the sequence input.

13. A method, comprising:
receiving, by a computing device, a selection of a class, wherein the class is a chimera creation class or a modification class;
receiving, by the computing device, a sequence input;
processing, by the computing device, the sequence input;
determining, by the computing device, a plurality of constraints based at least in part on the processed sequence input; and
designing, by the computing device, a primer based at least in part on the plurality of constraints.

14. The method of claim 13, further comprising:
installing, by the computing device, one or more sequence libraries; and
determining, by the computing device, the plurality of constraints based at least in part on the one or more sequence libraries.

15. The method of claim 14, wherein processing the sequence input further comprises comparing the sequence input to the one or more sequence libraries.

16. The method of claim 13, wherein the plurality of constraints includes at least one of a melting temperature, a primer length, and an overlap sequence.

17. The method of claim 13, further comprising generating, by the computing device, a report including information on the primer.

18. The method of claim 13, wherein determining a plurality of constraints further comprises:
receiving, by the computing device, a desired range of melting temperatures; and
designing, by the computing device, the primer based at least in part on the plurality of constraints and the desired range of melting temperatures.

19. The method of claim 13, wherein processing the sequence input further comprises:
splitting, by the computing device, the sequence input into two or more parts based at least in part on one or more symbols in the sequence input; and
generating, by the computing device, a protein translation into one or more frames.

20. The method of claim 13, designing the primer further comprises:
receiving, by the computing device, a desired range of melting temperatures;
calculating, by the computing device, a melting temperature value for one or more primers based at least in part on a plurality of nucleotides within the one or more primers;
adjusting, by the computing device, a length of one or more primers based at least in part on the calculated melting temperature value;
determining, by the computing device, the calculated melting temperature value is within the desired range; and
determining, by the computing device, one or more appropriate locations for the one or more primers based at least in part on the sequence input.


SEQUENCE LISTING (42 sequences)

SEQ ID NO: 1 - DNA, 15 bp, synthetic construct
  source 1..15: mol_type = other DNA
ggttctccaggttct

SEQ ID NO: 2 - DNA, 21 bp, synthetic construct
  source 1..21: mol_type = other DNA
ggttctccaggtccaagcggt

SEQ ID NO: 3 - DNA, 15 bp, synthetic construct
  source 1..15: mol_type = other DNA
ggtggcggtggttct

SEQ ID NO: 4 - DNA, 36 bp, synthetic construct
  source 1..36: mol_type = other DNA
ggttctgctggttctgctgctggttctggtgaattc

SEQ ID NO: 5 - DNA, 42 bp, synthetic construct
  source 1..42: mol_type = other DNA
ggttctccaggttctactttatctcgttggactggtcgttta

SEQ ID NO: 6 - DNA, 64 bp, synthetic construct
  source 1..64: mol_type = other DNA
tagaacctggagaacctttaactttttttttagtatcattcatataaataatatcatctaaagc

SEQ ID NO: 7 - DNA, 45 bp, synthetic construct
  source 1..45: mol_type = other DNA
tctccaggtccaagcggtactttatctcgttggactggtcgttta

SEQ ID NO: 8 - DNA, 67 bp, synthetic construct
  source 1..67: mol_type = other DNA
cgcttggacctggagaacctttaactttttttttagtatcattcatataaataatatcatctaaagc

SEQ ID NO: 9 - DNA, 42 bp, synthetic construct
  source 1..42: mol_type = other DNA
ggtggcggtggttctactttatctcgttggactggtcgttta

SEQ ID NO: 10 - DNA, 64 bp, synthetic construct
  source 1..64: mol_type = other DNA
tagaaccaccgccacctttaactttttttttagtatcattcatataaataatatcatctaaagc

SEQ ID NO: 11 - DNA, 53 bp, synthetic construct
  source 1..53: mol_type = other DNA
gttctgctgctggttctggtgaattcactttatctcgttggactggtcgttta

SEQ ID NO: 12 - DNA, 74 bp, synthetic construct
  source 1..74: mol_type = other DNA
gaaccagcagcagaaccagcagaacctttaactttttttttagtatcattcatataaataatatcatctaaagc

SEQ ID NO: 13 - DNA, 44 bp, synthetic construct
  source 1..44: mol_type = other DNA
gttctggtactattattggttgtggttttttattatctttagct

SEQ ID NO: 14 - DNA, 35 bp, synthetic construct
  source 1..35: mol_type = other DNA
ataatagtaccagaaccagaaccagaacctttaac

SEQ ID NO: 15 - DNA, 34 bp, synthetic construct
  source 1..34: mol_type = other DNA
aaatgactctcgagcaccaccaccaccaccactg

SEQ ID NO: 16 - DNA, 42 bp, synthetic construct
  source 1..42: mol_type = other DNA
tgctcgagagtcatttgtttagtaatataagcaacagaatca

SEQ ID NO: 17 - DNA, 51 bp, synthetic construct
  source 1..51: mol_type = other DNA
gatataccatgaaagaaaaattaaatcgtttattatttacttctaaaactc

SEQ ID NO: 18 - DNA, 49 bp, synthetic construct
  source 1..49: mol_type = other DNA
tctttcatggtatatctccttcttaaagttaaacaaaattatttctaga

SEQ ID NO: 19 - DNA, 46 bp, synthetic construct
  source 1..46: mol_type = other DNA
gttgcccgggctgctgttctgatacggacagaaaaataatctcagg

SEQ ID NO: 20 - DNA, 53 bp, synthetic construct
  source 1..53: mol_type = other DNA
cagcagcccgggcaacaacttgctatatattctgttaatgttatgctacattg

SEQ ID NO: 21 - DNA, 36 bp, synthetic construct
  source 1..36: mol_type = other DNA
ctgagatgtagtacacctctgggatttgatgaagta

SEQ ID NO: 22 - DNA, 41 bp, synthetic construct
  source 1..41: mol_type = other DNA
gtgtactacatctcagaataattccatgtacaccttctaat

SEQ ID NO: 23 - DNA, 41 bp, synthetic construct
  source 1..41: mol_type = other DNA
ttaatgtgtataatgacaactcctaaaaaaggattatgtgc

SEQ ID NO: 24 - DNA, 46 bp, synthetic construct
  source 1..46: mol_type = other DNA
tcattatacacattaagtatatcgtgttttcatttgtatcatatga

SEQ ID NO: 25 - DNA, 33 bp, synthetic construct
  source 1..33: mol_type = other DNA
catatggctctgaaaatacaggttttcgtgatg

SEQ ID NO: 26 - DNA, 24 bp, synthetic construct
  source 1..24: mol_type = other DNA
taaggatcctgcgagctctgtcga

SEQ ID NO: 27 - DNA, 46 bp, synthetic construct
  source 1..46: mol_type = other DNA
ctgtattttcagagccatatggtgaacaaaaccgaaaaaaccagcg

SEQ ID NO: 28 - DNA, 42 bp, synthetic construct
  source 1..42: mol_type = other DNA
gagctcgcaggatccttacggcagcagatcctgaccattatt

SEQ ID NO: 29 - DNA, 980 bp, synthetic construct
  source 1..980: mol_type = other DNA
ttttgtttaactttaagaaggagatataccatgggcagcagccatcatcatcatcatcacagcagcggcgagaatctttattttcagggccatatgaaagaaaaattaaatcgtttattatttacttctaaaactcgtatgcgtgttttttctaaattatctcgttatttatctaatggtgttcctgttacttttgctttagctgaattatataaatttacttctgatgaaggtcgtaaaaaagataatcctgatgcttttgctttacaacgttggttaattgctgttcgtaatggtaaaactttagctgaagctatgcgtggttgggttccttttgatgaattatctattatttctgctggtgaaatttctggtaatgttcatcaagctttagatgatattatttatatgaatgatactaaaaaaaaagttaaaggttctggttctggttctggtactttatctcgttggactggtcgtttacgtttattttttgatcgttttattccttggtctatttataaaactattattggttgtggttttttattatctttagcttctttaattaatgctggtattcctgttcctgaagctttacgtattattatgaaaactgcttctccttggtataaagaacgtttagttgctattcgttatcgtttattaaatggtgaacgtaatttaggtgaagctttaaattcttctggttatatttttccttctaaacaaatgattatggatttacgttcttatgctgctttaggtggttttgatgaaatgttaaataaattatctgttcaatggcaagatgattctgttgcttatattactaaacaaatgacttaactcgagcaccaccaccaccaccactgagatccggctgctaacaaagcccgaaaggaagctgagttggctgctgccaccgctgagcaataactagcataaccccttggggcctctaaacgggtcttgagggg

SEQ ID NO: 30 - DNA, 109 bp, synthetic construct
  source 1..109: mol_type = other DNA
aaaattgtgcgctcaatgtagcataacattaacagaatatatagcaagtggcggggggatatctgatacggacagaaaaataatctcaggtcatgaaacatctgtacga

SEQ ID NO: 31 - DNA, 122 bp, synthetic construct
  source 1..122: mol_type = other DNA
agagtgaaataaaaaaaagtggatcatttatctcatatgatacaaatgaaaacacgatatacttaatgtctataatgacaactcctaaaaaaggattatgtgcaaccatatttaatggggct

SEQ ID NO: 32 - DNA, 112 bp, Synthetic construct
  source 1..112: mol_type = other DNA
agagcccctgcccggtaaattagaaggtgtacatggaattattctgagatcaagtacacctctgggatttgatgaagtactctctatgattcaggattccagtggtattccc

SEQ ID NO: 33 - DNA, 202 bp, synthetic construct
  source 1..202: mol_type = other DNA
ccatgggccatcaccatcaccatcacgaaaacctgtattttcagagccatatggcgaattctgcggatcctgcgagctctgtcgacgcaaagcttctcgagggtacccggtagtggtagtggtagtgcttttggacataaaagtctcggtataccgcttaagacgcctaggacgctcgagacagctgcgtttcgaagagctc

SEQ ID NO: 34 - DNA, 321 bp, synthetic construct
  source 1..321: mol_type = other DNA
atggtgaacaaaaccgaaaaaaccagcgatctgatgtttgaacgctttaaacgtaacgtgagcgaaattgttaccggtgatggtggtgaactggaactgaccgttgaacagcgtaaatactttctgatcttcaaaaacggcgattttctggttagcagctgccatatgaaacatcatctggttcagatgctgcgtgaaattgcaacccgtaaaggttatccgaatctgaccatttatgaggtgaacctgaaagatattcgcctgctgtatgaagcaagcctgaaaaccgttcagaataatggtcaggatctgctgccgtaa

SEQ ID NO: 35 - DNA, 489 bp, synthetic construct
  source 1..489: mol_type = other DNA
ttggctgaagcgatgagggggtgggtaccttttgacgaactgtcaataatttccgccggggaaatctcaggtaatgttcaccaggcgttggatgatatcatttatatgaatgatacaaaaaagaaagtaaaagcgcactggcagggattatttatcctgtagtcctgcttctgacgacatgtctgtatttgcatatatttggaactcaggttgttccggcattttcaggcatcctgcctgtagagaaatggcagggcgcaggcaggactatgtattatcttgctgtattcgttcaggattatcttgtcattacactgctgtcttttatgatggtgatattattaatactggcaactctgtcaagatggaccggaaggttgcgtctcttctttgacagatttattccctggtcgatatacaaaaccattataggatgtggtttcttgttatcactggcatcgcttattaatgcaggtatccctgtaccgg

SEQ ID NO: 36 - DNA, 196 bp, synthetic construct
  source 1..196: mol_type = other DNA
actctgtcaagatggaccggaaggttgcgtctcttctttgacagatttattccctggtcgatatacaaaaccattataggatgtggtttcttgttatcactggcatcgcttattaatgcaggtatccctgtaccggaagccttacgaataataatgaaaacggcaagtccgtggtataaggaaagattagttgcta

SEQ ID NO: 37 - DNA, 196 bp, synthetic construct; identical to SEQ ID NO: 36
  source 1..196: mol_type = other DNA

SEQ ID NO: 38 - DNA, 168 bp, synthetic construct
  source 1..168: mol_type = other DNA
actctgtcaagatggaccggaaggttgcgtctcttctttgacagatttattccctggtcgatatacaaaaccattataggatgtggtttcttgttatcactggcatcgcttattaatgcaggtatccctgtaccggaagccttacgaataataatgaaaacggcaagt

SEQ ID NO: 39 - protein, 294 aa, synthetic construct
MGSSHHHHHHSSGENLYFQGHMKEKLNRLLFTSKTRMRVFSKLSRYLSNGVPVTFALAELYKFTSDEGRKKDNPDAFALQRWLIAVRNGKTLAEAMRGWVPFDELSIISAGEISGNVHQALDDIIYMNDTKKKVKGSGSGSGTLSRWTGRLRLFFDRFIPWSIYKTIIGCGFLLSLASLINAGIPVPEALRIIMKTASPWYKERLVAIRYRLLNGERNLGEALNSSGYIFPSKQMIMDLRSYAALGGFDEMLNKLSVQWQDDSVAYITKQMTLEHHHHHHDPAANKARKEAELA

SEQ ID NO: 40 - protein, 36 aa, synthetic construct
KLCAQCSITLTEYIASGGGISDTDRKIISGHETSVR

SEQ ID NO: 41 - protein, 41 aa, synthetic construct
ESEIKKSGSFISYDTNENTIYLHSIMTTPKKGLCATIFNGA

SEQ ID NO: 42 - protein, 37 aa, synthetic construct
EPLPGKLEGVHGIILRSSTPLGFDEVLSMIQDSSGIP